(19) United States
(12) Patent Application Publication
(10) Pub. No.: US 2021/0236320 A1
(43) Pub. Date: Aug. 5, 2021

(54) Methods for Installing Spacer for Non-weightbearing Cast

(71) Applicants: Ricky L. Jones, Little Elm, TX (US); Jake Preston Evans, Charlotte, NC (US)

(72) Inventors: Ricky L. Jones, Little Elm, TX (US); Jake Preston Evans, Charlotte, NC (US)

(21) Appl. No.: 16/777,089

(22) Filed: Jan. 30, 2020

(51) Int. Cl. A61F 5/01

(52) U.S. Cl. CPC A61F 5/0102; A61F 2005/0181; A61F 5/0111

(57) ABSTRACT

Methods for installing braces or spacers or braces that protect the heel of a person wearing a non-weightbearing cast, for applying multiple exemplary embodiments are described, such as an embodiment with two braces that are each attached to at least one point on either side of the foot, with one brace below the heel and the other behind the heel with a clearance space between each brace and the cast, to redirect loads from the heel to points behind the ankle and forward of the arch.

This specification claims the benefit of the prior filed provisional application No. 62/894,696.

### BACKGROUND OF THE INVENTION

The problem with loads on non-weightbearing casts is that the flesh of the heel easily develops ulcers when contact over 5 pounds is applied. Applicant has found no notable solutions to this problem in the prior art.

This inventor arrived at this design as an orthopedist, in a Technician's capacity, over the course of 30 plus years of experience. It is a necessary improvement to the art of cast making.

This disclosure provides the methods for installing such an apparatus, and similar nonlimiting exemplary apparatuses.

### BRIEF SUMMARY OF THE INVENTION

What is needed and not found in the prior art is a method to install devices that prevent heel sores in non-weightbearing casts. This is done through braces that both redirect common forces and create awkward obstructions that discourage certain improper placements of the cast.

This disclosure provides a preferred method and alternate methods. These methods teach a user to install various apparati which are not claimed as a part of this specification. The methods can be used to install similar apparati that accomplish the same objectives. It is imperative to clarify that the braced cast is still a non-weightbearing cast, and that routine loading is still limited to nearly zero loading.

About the Apparatus and Similar Conceptual Nonlimiting Apparatuses

In the specification, the preferred loading of the casts discussed is zero pounds. However, to accommodate loads up to approximately 5 pounds, this method installs types of bracing that are helpful. LOADING OVER 5 POUNDS IS IN NO WAY IMPLIED OR RECOMMENDED BY THIS METHOD.

The solution herein disclosed redirects common axial forces from the heel area to points of contact above and points of contact forward of the heel. The primary embodiment includes two braces that protect the bottom and back of the heel from contacts. In an embodiment, the braces are secured to a finished leg cast. Securing is done by means that do not compromise the integrity of the finished cast by using fiberglass rolls, plaster or other methods familiar to those in the field of orthopedics.

The solution also discourages placement of the non-weightbearing cast in two general and improper positions. First is resting the weight of the body on the cast. The placement of a brace below the heel creates awkward, unbalanced loading on the cast and leg. Additional features are contemplated such as installing uneven braces, creating further awkwardness. Similarly, installing a brace behind the foot creates another awkward loading when resting the cast horizontally, with the toes up.

In this specification the word brace is considered synonymous with other terms used herein, including “bracket” and “spacer”. The finished assembly, produced using this method is also described as a “braced non-weightbearing cast”.

In this specification the concept of a cast is used as an exemplary application. One of ordinary skill in the art would see that this method would apply to bracing other non-weightbearing applications such as splints.

Common loading refers to loads that many cast wearers would apply to the heel in normal activities for an injured person. For example a forward force directed from the heel area toward the toes during the frequent placement of the cast horizontally aligned and resting on a horizontal surface—picture a coffee table. Other common loads include resting some amount of the wearer's bodyweight on a non-weightbearing cast, in a generally vertical loading where the ground contacts the cast at the heel, and applies a reactionary force upward along the leg and cast.

In the preferred embodiment, two braces are used on a single non-weightbearing leg cast. As a nonlimiting embodiment, any number of braces can be used from at least one, and up to an integral cap (also described herein).

In a nonlimiting embodiment, each brace is attached at 4 points including two points on the left side and two on the right. With multiple attachment points, it is possible to redirect common forces to any “best” part of the cast using eccentric torque loading instead of single mounting attachments. That is to say, the original design directs all forces to each point of attachment, with one attachment on either side. A better place to redirect a load is to the back of the ankle—along the Achilles. To redirect the load, the rear brace attaches with at least two points on either side of the ankle for at least 4 points total. Thereby, common forces would impact the brace and create a torque instead of a direct impact. To be clear, a single, common force applied to the cast in a direction toward the heel, would in turn be applied “off center” to multiple eccentrically located attachments, to create a torque around the braced cast assembly.

In an embodiment, there are two attachment points on each side, in the ankle area. The first attachment point is lower than the other—one toward the sole of the foot and the other higher in the ankle area—perhaps ½″ apart. The attachment points are also slightly (e.g. ¼″) offset forward and rearward. With this 4 point mounting, any common load directed toward the back of the heel would be received by the multiple attachment points unevenly—eccentrically. This would cause a torque and apply the force at the back of the leg or on the sole of the foot. With an alternative positioning of the 4 mounting points a common force and resulting torque could be applied wherever is best.

An alternative embodiment that is installed by the method herein disclosed comes from the objective of the device—to prevent contact at the heel. The idea is to add an audible “click” at the brace to notify the wearer that a load of more than 5 pounds has been experienced by the cast. The click should reset—spring loaded perhaps—so that it is repetitive, notifying the wearer of his/her many 5# overloads. The intent is to be disruptive, and to create an incentive to avoid overloading the cast.

Another alternative embodiment speaks to children—who are notorious for overloading the cast at the heel. It is important to reiterate that: LOADING OVER 5 POUNDS IS IN NO WAY IMPLIED ENCOURAGED OR RECOMMENDED BY THIS METHOD. The suggestion is to install attachments or to install reshaped braces with decorative and aesthetically appealing, but functional brace designs. As an example, floral shapes of suitable size, shape and material can be attached as part of the method. As another example, the braces themselves may be formed from the supplier with pair of ears shape and a tail shape. In another example, the reshaping elements are formed and attached to the braces at the factory, or the same elements can be attached to the braces by the practitioner.

Another embodiment continues to use electronics that would allow both notice that an overload has occurred and there is an aesthetic advantage for younger wearers. Again: LOADING OVER 5 POUNDS IS IN NO WAY IMPLIED, ENCOURAGED OR RECOMMENDED BY THIS METHOD.
a. in an embodiment, Electronics include switches mounted on the braces to activate upon a 5# overload, with red LED indicators that flash for 5 seconds when an overload condition is momentarily present.
b. Electronics can include a sonolert—an audible alarm to indicate an overload condition. The alarm can be a click, for subtlety. The alarm can be a garish siren.

In an embodiment, Other electronics include a molded pressure pad with at least one pressure sensor that respond to 5 # loading. Schmersal sms4-500-500 is an exemplary pressure mat with about 244 sensors. This would be overkill, but offers the concept of a low profile mat with multiple input sensors. In this application, only 4 to 6 sensors are needed in a minimal arrangement.

In an embodiment, in place of the braces, a single molded cap is installed using the methods herein disclosed, and used as a shield around the entire heel area. Again: LOADING OVER 5 POUNDS IS IN NO WAY IMPLIED, ENCOURAGED OR RECOMMENDED BY THIS METHOD. The cap would secure to the cast similar to the braces—at two opposite sides of the heel. The cap, however could be closer to the cast than ¼″ previously outlined. The installer of the cap can install a seal around the edges of the cap, to seal the cap to the surface of the cast. This seal can also be made of a rigid material sufficient to create a ring of contact between the cap and the cast where the redirected forces would be applied along a sizeable contact area. In an embodiment, the method includes installing caps that have electronic switches and lighted outputs that mount and operate similar to those previously discussed. A familiar and similar concept is used to protect a broken nose from contact (often used for basketball). In this exemplary concept, the mask redirects any forces away from the nose area and to other parts of the face. One of ordinary skill in the art would be familiar with materials appropriate to the application, including but not limited to plastics, carbon fiber and metal.

In an embodiment, a direct force testing system is employed and installed using the disclosed method. This embodiment would be useful as a test mechanism or in applications that required the accurate measurement of the forces applied to a cast and in turn applied to the leg and foot of the patient. Force sensing pads (for example a pair of pads electronically connected by a normally open switch) are used at force transfer contact points and molded onto the cast by applying an additional pad at the places where the load is more optimally applied. The concept of a broad, soft pad at the heel was rejected because it could result in undesired contact and a resulting ulcer. However, the top of the foot, the bottom sole of the foot and the back of the ankle are mentioned herein as good places to direct/redirect forces. At these locations an applied force can be measured properly. An installer places the force pad is placed in contact with the cast (or even inside the cast to the cast liner or to the foot/leg) at the best points for measuring redirected contact forces—and then secures to the completed cast as needed. Force pad readings are communicated using direct wiring or externally mounted wireless devices.

Certain nonlimiting embodiments are outlined herein, and one of ordinary skill in the art would be aware of modifications or alternatives that would be similar. One of ordinary skill in the art would also be aware of tools and hardware to aid in the installation process, and would use such items to brace, stabilize, locate, prepare, install, secure and similarly apply the methods herein disclosed.

### BRIEF DESCRIPTION OF THE DRAWINGS

FIG. 1 is a nonlimiting demonstration of a cast being applied to a right leg, typical of either leg.

FIG. 2 is a nonlimiting demonstration of the brace installation.

FIG. 3 demonstrates a nonlimiting alternative mounting detail concept using 2 fastening points.

FIG. 4 is a nonlimiting demonstration of a warning label. The information on the label is necessary for effective use of a braced non-weightbearing cast.

FIG. 5 is a nonlimiting demonstration of other brace embodiments for which the methods herein would apply.

FIG. 6 is a nonlimiting demonstration of a heel shield.

FIG. 7 is a nonlimiting demonstration of a braced non-weightbearing cast resting on a pillow and table surface.

FIG. 8 is a nonlimiting demonstration of a braced non-weightbearing cast from a view below the cast and foot.

### DETAILED DESCRIPTION OF THE PREFERRED EMBODIMENT

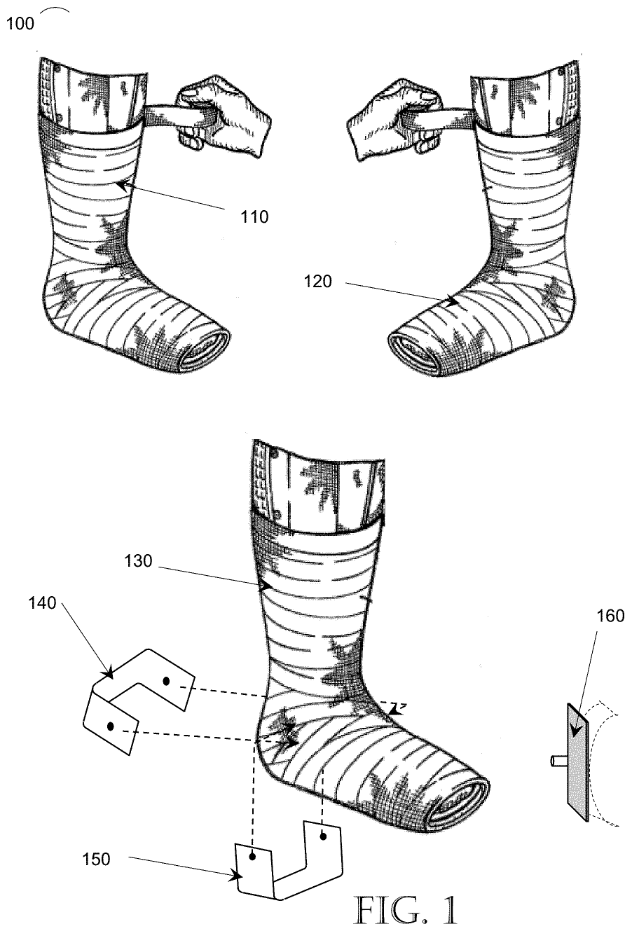

FIG. 1 is a nonlimiting demonstration of a cast being applied to a right leg, typical of either leg. The figure 100 shows the installation using typical fiberglass tape as a component of an exemplary 110 (right) or 120 (left) fiberglass non-weightbearing cast. 130 represents a finished non-weightbearing cast, having 140 rear and 150 lower braces and a general location of the braces. The figure also shows 160, a nonlimiting conceptual mounting detail, right shown and left typical. Such 160 mounting details are installed during the cast fabrication process and secured with fiberglass tape or other appropriate means. 160 also demonstrates a nonlimiting flexible backing plate that would provide a securing surface for attaching the backing plate to the 130 cast. 140 and 150 braces would then mount to the 160 mounting details; and would then be further secured using means such as fiberglass casting tape or plaster.

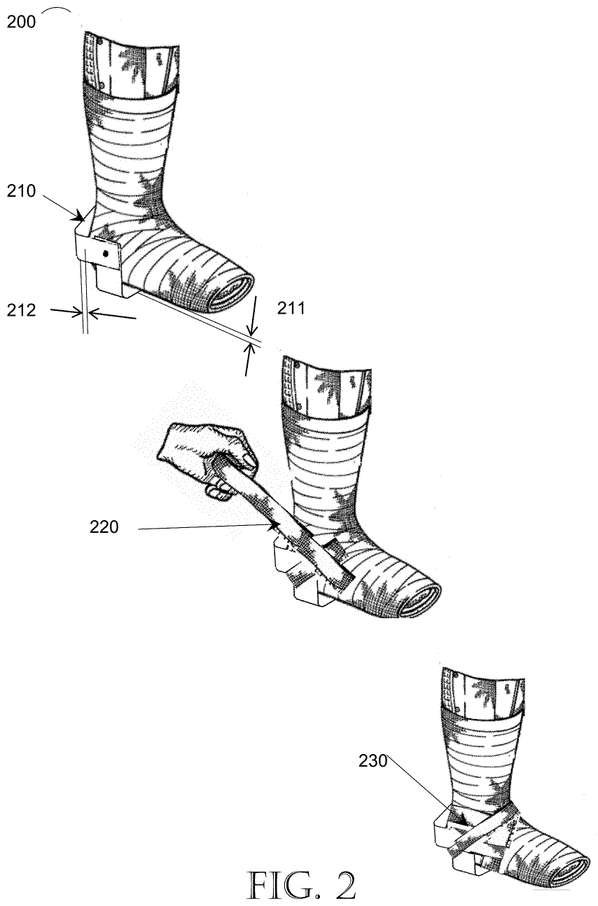

FIG. 2 is a nonlimiting demonstration of the brace installation. 200 shows the process of locating 210 (rear shown, lower typical) brace being located to leave a 211 and 212 minimum spacing such as 0.25 inches. 211 and 212 spacings being between the inner surface of the brace and the outer surface of the cast. Once located, braces are then 220 secured with fiberglass tape. 230 demonstrates a secured pair of braces. Further application of fiberglass tape is contemplated to make an integrated and uniform appearance of the braces and cast.

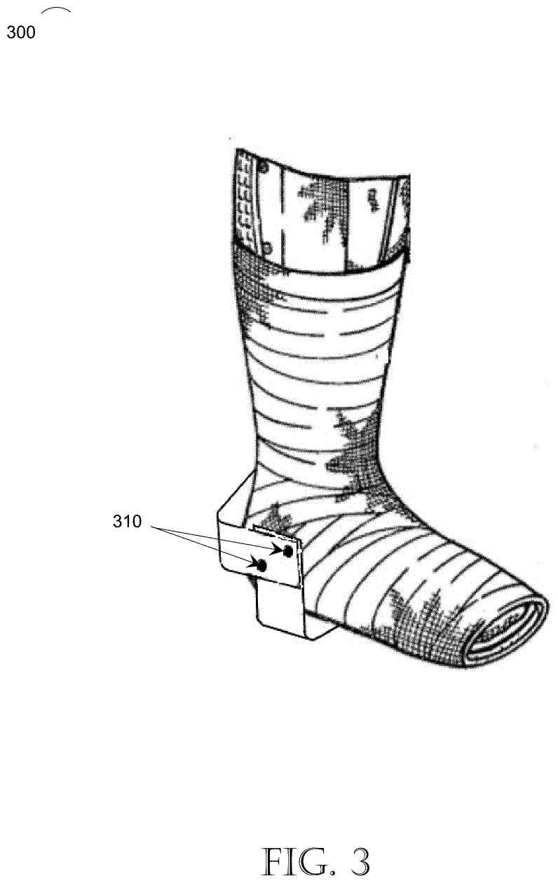

FIG. 3 demonstrates an a nonlimiting alternative mounting detail concept using 2 fastening points. 300 demonstrates an assembly view of two braces on one cast using a total of four anchoring points. 310 locating points are shown in a nonlimiting arrangement as circles to represent a fastening method such as a female fastener attached to a backing plate that is then mated with a male fastener that extends through each of the 310 (2 shown typ 4) locating points (clearance holes).

In the 300 attachment concept, application of forces is usually along eccentric axes. When these forces are applied, there is a resulting torque. This torque, in turn realigns and displaces the force to impact points that are remote from the heel. As a result, the installer of this 4 point brace should be aware of the resulting impact points and may provide padding or reinforcement to accommodate the resulting forces.

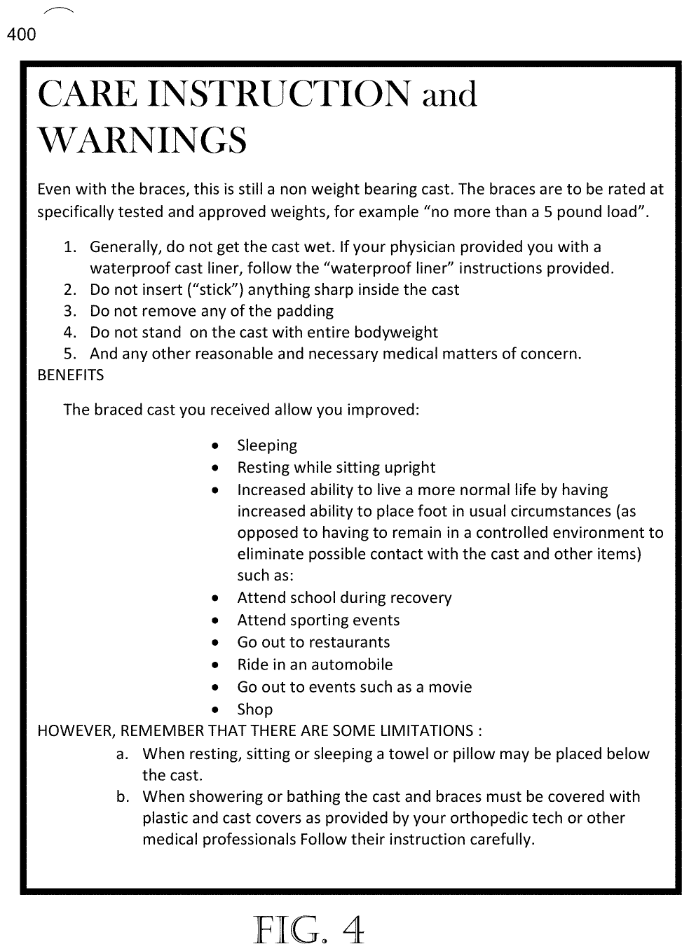

FIG. 4 is a nonlimiting demonstration of a warning label. The information on the label is necessary for effective use of a braced non-weightbearing cast. The method for installing such braces must include a clearly understood warning of how to properly use the braced cast. This includes a mechanical clicking alert that is activated when forces above 5# are applied. For this type of bracing the mechanism should be tested before and after installation using a load of an appropriate weight, for example a 5# weight. The reset mechanism should also be tested. For an embodiment that utilizes decorative features on the braces similar tests should be provided. The mechanism should be demonstrated for the user, parents or other available responsible person.

For embodiments using electrical features, battery holders must be installed and secured by methods obvious to anyone familiar with the art. For this type of bracing the mechanism should be tested before and after installation using a load of an appropriate weight, for example a 5# weight. The reset mechanism should also be tested. The mechanism should be demonstrated for the user, parents or other available responsible person.

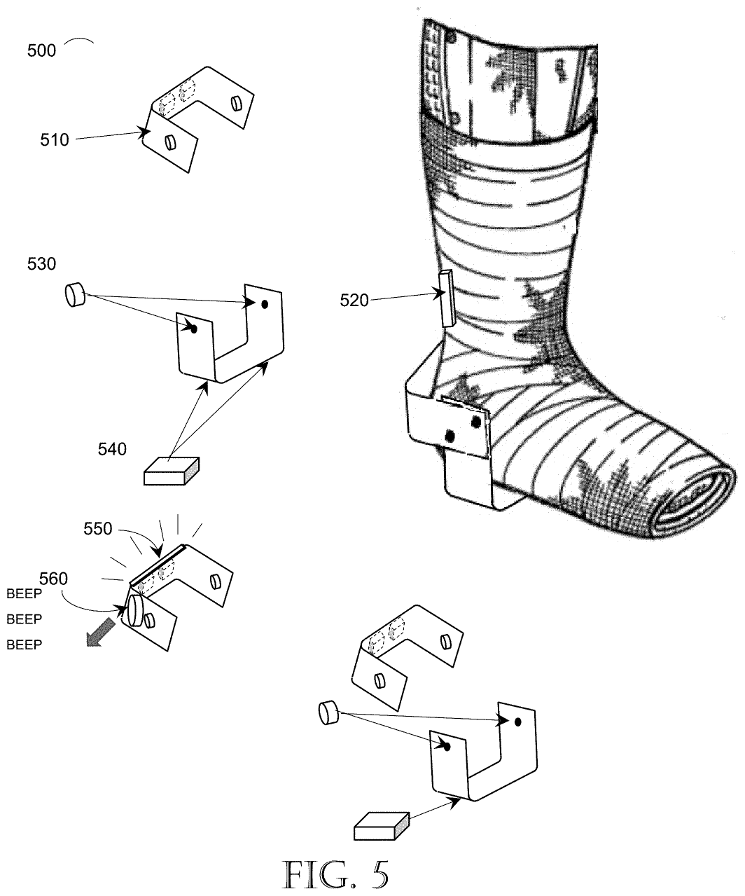

FIG. 5 is a nonlimiting demonstration of other brace embodiments for which the methods herein would apply. 500 presents 2 concepts. 510 rear brace is typical, and uses 520 battery pack secured to the cast or similar and optionally secured using fiberglass tape or similar. 530 nonlimiting anchoring mechanism is installed on each anchor to utilize pressure sensitive contacts to connect positive and negative leads (not shown) and thus complete circuits activating 550 lights and 560 audible alerts. In another nonlimiting embodiment 540 pressure pads are secured to the 510 typ brace to receive user contact to close circuits and activate a combination of 540 visual alerts and 550 audible alerts. The components demonstrated in 500 are designed to indicate to the user that a force near or over 5 pounds has occurred and contacted at least one of the braces.

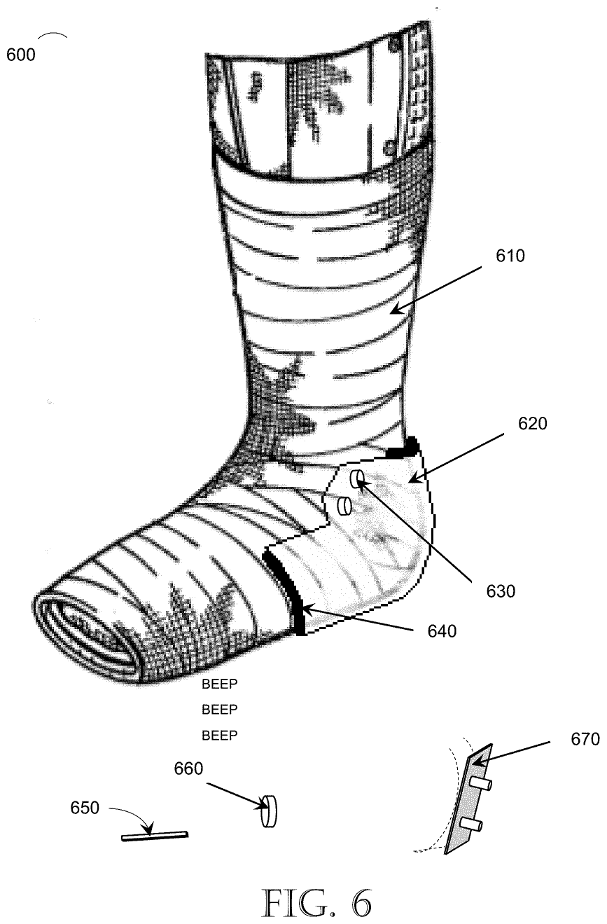

FIG. 6 is a nonlimiting demonstration of a heel shield. The 620 shield is attached to the 610 non-weightbearing cast using methods similar to the rear and lower brace methods demonstrated in this specification. However, multiple 630 anchoring details are most appropriate. This embodiment presents the opportunity for 640 contact pads to be used at the points of transferred impact forces to specific areas of the foot and heel. Such contact pads fit inside the previously mentioned gaps between braces and the non-weightbearing cast. 670 anchoring plates having more than one anchor mechanism are an example. Additionally, circuitry and components such as 650 visible lights and 660 audible sonolerts are installed and used by the patient to give notice of contact loads near or greater than 5 pounds.

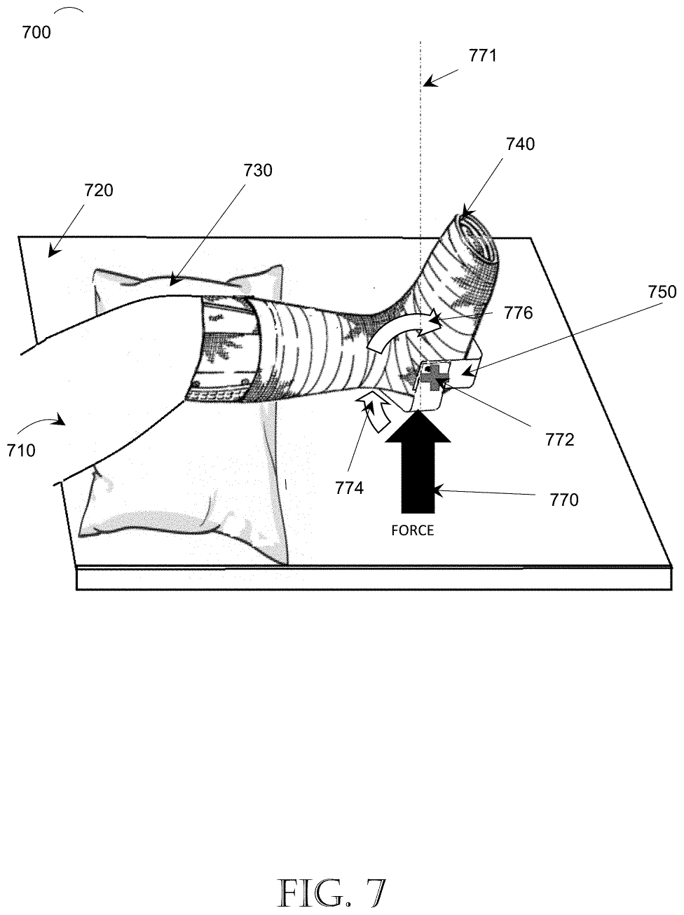

FIG. 7 is a nonlimiting demonstration of a braced non-weightbearing cast resting on a pillow and table surface. In the FIG. 700, a leg 710 is shown generally resting as recommended on a 720 table surface. The 730 pillow supports the leg below the knee area for this type of 740 non-weightbearing cast. The 750 braces redirect forces that would normally be applied to the heel area of the 740 cast. The forces are now taken from contact point with the 750 braces and applied to the 740 cast through the attachment means. Here, a 770 force is applied to the cast by the table, caused by the weight of the leg and cast resting on said table. This force is generally along a 771 vertical axis direction. Elsewhere in this specification such a force is one of many common forces. The force is experienced by the 750 brace assembly; and transferred about the 772 center of the brace attachment, generally to points 774 near the ankle and 776 top of the foot.

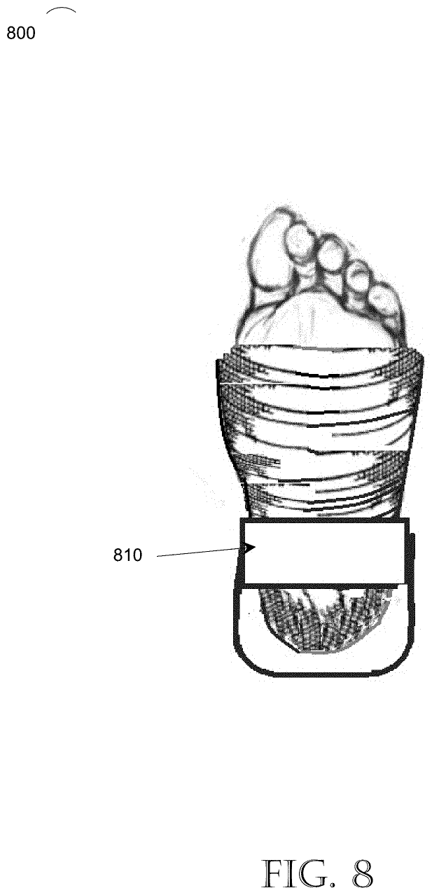

FIG. 8 is a nonlimiting demonstration of a braced non-weightbearing cast from a view below the cast and foot. The brace 810 is shown with a space between the brace and the back of the heel.

The drawings provided are exemplary to demonstrate the details shown. The shapes, orientations, materials, modifications, alternatives and substitutions that are obvious to one of ordinary skill in the art are included. Further, tools and alternative fastening systems are also contemplated by the inventor and this specification. Finally, the shape of the braces are contemplated as rounded, eccentric, triangular, and others.

I claim:

1. A method for installing braces to a non-weightbearing cast by an installer to a patient whereby
a doctor or provider orders non-weightbearing casts of any of the type
Fiberglass long leg cast,
short leg cast,
Fiberglass long leg splints,
short leg splints.
Plaster long leg cast,
short leg cast,
Plaster long leg splints,
short leg splints.
Hip Spica Cast
long leg Fiberglass and Plaster,
or other similar types of casts;
to aid in healing common fractures of the type:
Tibia, Fibula, Ankle, Navicular, Cuboid, Cuneiform Bones, Stress Fractures;
an installer fabricates and installs an appropriate non-weightbearing cast to the patient;
installer clearly communicates to the patient that the objective is to protect the heel from contact when a force is applied to the cast at the heel area,
installer selects appropriate braces,
and that this is accomplished by redistributing the force to the distal and proximal ends of the foot and ankle structures;
installer opens the packaging which includes at a minimum, the brace assembly (or two braces) and their attachment hardware;
Installer attaches the hardware and brace assemblies to the cast,
during or after which the cast is completely formed;
as the braces are placed, a first brace is to be located at the distal end of the foot and cast, with attachments at the inner side of the navicular and outer side of the cuboid bones;
a second brace is located to the posterior end of the foot and cast, with attachments at the inner side of the medial malleous bone and outer side of the lateral malleolus bone;
installer holds the brace in place and secures using 2 inch or 3 inch width fiberglass cast material;
installer smooths and molds the cast material to secure the braces firmly and smooth edges for uniformity.

2. The method of claim 1 further including steps for after both braces are secured and molded into place, including providing
specific care instructions that are necessary and include appropriate warnings;
once patient is clearly informed of the necessary care inform patient of the benefits of the braced cast over the nonbraced cast;
a. Sleeping
b. Resting
c. Increased ability to live a more normal life by having increased ability to place foot in usual circumstances (as opposed to having to remain in a controlled environment to eliminate possible contact with the cast and other items) such as:
Attend school during recovery,
Attend sporting events,
Go out to restaurants,
Ride in an automobile,
Go to movie,
Shop,
And any other activities beyond historical restrictions;
provide further traditional instructions for resting, sitting or sleeping using a towel or pillow that may be placed below the cast; and
when showering or bathing the cast and braces must be covered with plastic and cast covers as provided by your orthopedic tech or other medical professional, following their instruction carefully.

3. The method of claim 1 further including steps specifically required to install other embodiments of braces with different features and capabilities; and
Testing of proper functionality;
of a nonlimiting mechanical, audible embodiment, wherein each mechanism is tested by the installer using an appropriate weighting to test for a 5# maximum load;
and of the electrical embodiments wherein the electrical connections and mechanisms are tested for operability; and
installing general items including batteries, attaching decorative features, and the like.
